(19) United States
(12) Patent Application Publication
(10) Pub. No.: US 2013/0259902 A1
(43) Pub. Date: Oct. 3, 2013

(54) CARRIER MATERIAL FOR BONE FORMING CELLS

(71) Applicants: Ying Fan, Perth (AU); Ming-Hao Zeng, Perth (AU)

(72) Inventors: Ying Fan, Perth (AU); Ming-Hao Zeng, Perth (AU)

(21) Appl. No.: 13/127,043

(22) PCT Filed: Nov. 4, 2009
(86) PCT No.: PCT/AU09/01435

§ 371 Date: Apr. 22, 2013

(30) Foreign Application Priority Data
Nov. 4, 2008 (AU) 2008905715
Feb. 13, 2009 (AU) 2009900622

(51) Int. Cl. A61L 27/54; A61L 27/36; A61L 27/38

(52) U.S. Cl. CPC A61L 27/54; A61L 27/3821; A61L 27/3608; A61L 27/3834
USPC 424/400; 424/93.7

(57) ABSTRACT

The present invention relates to a carrier material and bone forming cells, which material is capable of providing treating orthopaedic conditions. In particular, the present invention relates to a bone forming carrier material comprising an anti-inflammatory amount of allogenic bone gel and a bone forming amount of bone-forming cells.

### FIELD

The present invention relates to methods for treating inflammation associated with bone, joint or connective tissue. In particular the present invention relates to a carrier material comprising allogenic bone gelatin (ABG) and bone forming cells, which material is capable of providing anti-inflammatory activities as well as bone cell growth supporting properties.

### BACKGROUND

In recent years the use of implantable material has increased dramatically in the field of orthopaedics. There is a variety of apparatus and methods for reducing, fixing and generally assisting the healing of fractured or grafted bone using these implantable materials. However, recognition of implants as foreign bodies by the immune system can often trigger the recruitment of killer cells to their host tissue interface leading to tissue inflammation, unwanted cell growth, aseptic loosening of joint implants or rejection. Thus, one of the most significant factors in the success or failure of orthopaedic surgery is the effect of general and local inflammation as well as bone loss.

Inflammation is traditionally treated with anti-inflammatory, analgesic, and/or anti-pyretic drugs, which form a heterogeneous group of compounds, often chemically unrelated, which nevertheless share certain therapeutic actions and side-effects. Corticosteroids represent the most widely used class of compounds for the treatment of the inflammatory response. Proteolytic enzymes represent another class of compounds which are thought to have anti-inflammatory effects. Hormones which directly or indirectly cause the adrenal cortex to produce and secrete steroids represent another class of anti-inflammatory compounds. A number of non-hormonal anti-inflammatory agents have been described. These agents are generally referred to as non-steroidal anti-inflammatory drugs (NSAIDS). Among these, the most widely used are the salicylates. Acetylsalicylic acid, or aspirin, is the most widely prescribed analgesic-antipyretic and anti-inflammatory agent. Examples of steroidal and non-steroidal anti-inflammatory agents are listed in the Physicians Desk Reference, 54^{th} Edition, 2000 (see pp. 202 and 217 for an index of such preparations).

To date, the majority of these anti-inflammatory agents are administered by traditional routes such as subcutaneous, intravenous or intramuscular injections. In recent times a number of authors have reported the delivery of anti-inflammatory or therapeutic agents directly to sites of orthopaedic surgery. However, the vast majority of these reports failed to report that many of these trials failed to provide adequate benefits to the patient as the agents used defused too rapidly from the wound site or in some cases exacerbated the inflammation.

Thus, there is a continuing need for methods and materials for treating inflammation, especially associated with orthopaedic conditions.

In addition to the problems with inflammation highlighted above it is also well known that orthopaedic surgery and its associated inflammation can also lead to bone loss. While a number of authors have suggested implanting bone forming cells to reduce and/or correct the bone loss, to date there have been no useful implantable material that provides a useful scaffold for correcting bone loss, while also treating inflammation.

Accordingly, there is a need for an implantable carrier material that is capable of providing anti-inflammatory properties while also providing a material for bone forming cells to attach and grow.

### SUMMARY

The inventors have previously developed a method of producing insoluble bone gelatin (see US Pat. Applc. No. 20030065392 to Zheng et al.) useful in lumbar fusion surgery. However, they have surprisingly discovered that a modified form of the insoluble bone gelatin produced by the method of Urist and colleagues termed herein allogenic bone gel (ABG) is anti-inflammatory per se, which is capable of overcoming or at least alleviating the problems identified above. Moreover, the ABG is capable of providing an implantable material for the controlled-release of other agents in situ and forms a superior material for growing bone-forming cells, in particular stem cells and osteoblasts.

Accordingly, in a first aspect, the present invention provides a bone forming carrier material comprising an anti-inflammatory amount of allogenic bone gel and a bone forming amount of bone-forming cells.

It will be appreciated by those skilled in the art that the four classic symptoms of inflammation are redness, elevated temperature, swelling, and pain in the affected area. Therefore, the implantable material of the present invention is suitable for inhibiting one or more of these four symptoms of inflammation. The implantable material is also suitable for inhibiting the influx of polymorphonuclear leukocytes (PMNs) into a tissue involved in inflammation.

In a second aspect the present invention provides a bone forming carrier material comprising allogenic bone gel, which gel provides at least a 3 fold reduction in the number of polymorphonuclear leukocytes in a subject's tissue and a bone forming amount of bone-forming cells.

In a third aspect the present invention provides a medical device coated with a bone forming carrier material of the first and second aspects.

In some aspects, the inflammation being treated is cytokine-induced inflammation. In other aspects, the inflammation is associated with bone disorders such as osteolysis.

It will be appreciated that the bone forming carrier material of the present invention substantially comprises allogenic bone gel per se. For example, the implantable material preferably includes at least 15% (w/w) allogenic bone gel (ABG). Desirably, the implantable material comprises at least 15%, 20%, 25%, 30%, 35%, 40%, 45%, 50%, 55%, 60%, 65%, 70%, 75%, 80%, 85%, or even 90% (w/w) ABG. However, the bone forming carrier material of the invention optionally includes a supplementary material selected from bioerodible materials (e.g., biodegradable and bioresorbable materials) and non-erodible materials. Bioerodible materials include polysaccharides, nucleic acids, carbohydrates, proteins, polypeptides, poly(α-hydroxy acids), poly(lactones), poly(amino acids), poly(anhydrides), poly(orthoesters), poly (anhydride-co-imides), poly(orthocarbonates), poly(α-hydroxy alkanoates), poly(dioxanones), poly(phosphoesters), or copolymers thereof. Desirably, the bioerodible material includes collagen, glycogen, chitin, starch, keratins, silk, hyaluronic acid, poly(L-lactide) (PLLA), poly(D,L-lactide) (PDLLA), polyglycolide (PGA), poly(lactide-co-glycolide (PLGA), poly(L-lactide-co-D, L-lactide), poly(D,L-lactide-co-trimethylene carbonate), polyhydroxybutyrate (PHB), poly(s-caprolactone), poly(δ-valerolactone), poly(γ-butyrolactone), poly(caprolactone), or copolymers thereof. Non-erodible materials include dextrans, celluloses and cellulose derivatives (e.g., methylcellulose, carboxy methylcellulose, hydroxypropyl methylcellulose, and hydroxyethyl cellulose), polyethylene, polymethylmethacrylate, carbon fibers, poly(ethylene glycol), poly(ethylene oxide), polyvinyl alcohol), poly(vinylpyrrolidone), poly(ethyloxazoline), poly(ethylene oxide)-co-poly(propylene oxide) block copolymers, poly(ethylene terephthalate)polyamide, or copolymers thereof. Bioerodible and non-erodible materials can be selected to introduce porosity or modify physical properties, such as strength and viscosity.

The bone forming carrier material of the invention optionally includes a biologically active agent. Biologically active agents that can be used in the compositions and methods described herein include, without limitation, osteogenic proteins, antibiotics, polynucleotides, anti-cancer agents, growth factors, and vaccines. Osteogenic proteins include, without limitation, BMP-2, BMP-3, BMP-3b, BMP-4, BMP-5, BMP-6, BMP-7, BMP-8, BMP-9, BMP-10, BMP-11, BMP-12, BMP-13, BMP-14, BMP-15, BMP-16, BMP-17, and BMP-18. Biologically active agents also include alkylating agents, platinum agents, antimetabolites, topoisomerase inhibitors, antitumor antibiotics, antimitotic agents, aromatase inhibitors, thymidylate synthase inhibitors, demineralized bone matrix, DNA antagonists, farnesyltransferase inhibitors, pump inhibitors, histone acetyltransferase inhibitors, metalloproteinase inhibitors, ribonucleoside reductase inhibitors, TNF alpha agonists, TNF alpha antagonists, endothelin A receptor antagonists, retinoic acid receptor agonists, immuno-modulators, hormonal agents, antihormonal agents, photodynamic agents, and tyrosine kinase inhibitors.

Accordingly, in a fourth aspect, the present invention provides a bone forming carrier material comprising at least 15% w/w allogenic bone gel, a bone forming amount of bone-forming cells, at least one supplementary material and at least one biologically active agent, wherein said biologically active agent supplements the anti-inflammatory effect of the allogenic bone gel.

In some embodiments, the biologically active agents are BMP2 and/or OP1.

In a fifth aspect the present invention provides a bone forming carrier material consisting essentially of allogenic bone gel, a bone forming amount of bone-forming cells and bone-morphogenetic protein-7 (OP-1) and/or bone-morphogenetic protein (BMP)-2.

In a sixth aspect, the present invention provides as method of treating an orthopeadic condition comprising the step of administering a bone forming amount of a bone forming carrier material comprising an anti-inflammatory amount of allogenic bone gel and a bone forming amount of bone-forming cells.

### BRIEF DESCRIPTION OF THE FIGURES

FIG. 1 shows the histo-pathological samples from sham operation+saline injection group.

FIG. 2 shows the histo-pathological samples from sham operation+LPS injection group.

FIG. 3 shows the histo-pathological samples from the allogenic bone gel+PLS injection group

FIG. 4 shows the histo-pathological samples from the Lycoll™+LPS group.

FIG. 5 shows H & E staining of co-culture of ABG scaffold with MSCs/DXM at 1, 2, 3 or 4 weeks.

FIG. 6 shows H & E staining of co-culture of milled bone with MSCs/DXM at 1, 2, 3 or 4 weeks.

FIG. 7 shows H & E staining of co-culture of ABG with MSCs/BMP-2 at 1, 2, 3 or 4 weeks.

FIG. 8 shows H & E staining of co-culture of milled bone with MSCs/BMP-2 at 1, 2, 3 or 4 weeks.

FIG. 9 shows the pattern of osteoblast special genes Col I, OP and OC expression during co-culture of ABG with MSCs/DXM.

FIG. 10 shows the pattern of osteoblast special genes Col I, OP and OC expression during co-culture of milled bone with MSCs/DXM.

FIG. 11 shows the pattern of osteoblast special genes Col I, OP and OC expression during co-culture of ABG with MSCs/BMP-2.

FIG. 12 shows the pattern of osteoblast special genes Col I, OP and OC expression during co-culture of milled bone with MSCs/BMP-2.

FIG. 13 shows the volume of new bone formed in spinal fusion experiments using autograph bone from the iliac crest (“Autograph”), allogenic bone gel (“ABG”), autologous bone forming cells (“BFCs”) and autologous bone forming cells mixed with allogenic bone gel (“ABG+BFCs”). New bone formation was measured at two postoperative time points, six weeks (upper panels) and ten weeks (lower panels), by micro CT scanning.

### DETAILED DESCRIPTION

Before describing the present invention in detail, it is to be understood that this invention is not limited to particularly exemplified methods and may, of course, vary. It is also to be understood that the terminology used herein is for the purpose of describing particular embodiments of the invention only, and is not intended to be limiting which will be limited only by the appended claims.

All publications, patents and patent applications cited herein, whether supra or infra, are hereby incorporated by reference in their entirety. However, publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

The practice of the present invention will employ, unless otherwise indicated, conventional techniques of cell biology, cell culture, molecular biology, and recombinant DNA, which are within the skill of the art. Such techniques are described in the literature. See, for example, Bailey & Ollis, 1986, “Biochemical Engineering Fundamentals”, 2nd Ed., McGraw-Hill, Toronto; Coligan et al., 1999, “Current protocols in Protein Science” Volume I and II (John Wiley & Sons Inc.); “DNA Cloning: A Practical Approach”, Volumes I and II (Glover ed., 1985); Handbook of Experimental Immunology, Volumes I-IV (Weir & Blackwell, eds., 1986); Immunochemical Methods in Cell and Molecular Biology (Mayer and Walker, eds., Academic Press, London, 1987), Methods in Enzymology, Vols. 154 and 155 (Wu et al. eds. 1987); “Molecular Cloning: A Laboratory Manual”, 2^{nd} Ed., (ed. by Sambrook, Fritsch and Maniatis) (Cold Spring Harbor Laboratory Press: 1989); “Nucleic Acid Hybridization”, (Hames & Higgins eds. 1984); “Oligonucleotide Synthesis” (Gait ed., 1984); Remington's Pharmaceutical Sciences, 17^{th} Edition, Mack Publishing Company, Easton, Pa., USA.; “The Merck Index”, 12^{th} Edition (1996), Therapeutic Category and Biological Activity Index; and “Transcription & Translation”, (Hames & Higgins eds. 1984).

It must be noted that as used herein and in the appended claims, the singular forms “a,” “an,” and “the” include plural reference unless the context clearly dictates otherwise. Thus, for example, a reference to “a protein” includes a plurality of such proteins, and a reference to “an agent” is a reference to one or more agents, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

In its broadest aspect the present invention encompasses a bone forming carrier material comprising an allogenic bone gel and bone forming cells, which, on implantation, reduces inflammation and provides a scaffold in which the bone forming cells proliferate.

The term “allogenic bone gel” (ABG) as used herein refers to a modified form of “insoluble bone gelatin” (ISBG) as compared to the ISBG produced by Urist and others, which can be prepared by the methods disclosed herein to produce a material that has anti-inflammatory properties and provides a microenvironment suitable for the growth and differentiation of bone forming cells. The allogenic bone gel generally comprises bone morphogenic protein (BMP), fibroblast growth factors (FGF), transforming growth factor beta (TGF-β), and growth factor binding proteins eg insulin-like growth factor (IGF) and BMP binding protein and any combination thereof. In particular, the ABG of the present invention has the features shown in the properties section of Table 1.

“Insoluble bone gelatin” is a product produced from demineralised bone matrix (BDM). BDM has been readily available for over ten years and is essentially milled (powdered) bone that has been treated with acid and/or EDTA to demineralise the bone i.e. remove calcium and/or phosphate while retaining lipids, collagen and non-collagenous proteins, including growth factors. The term “DBM” is well understood in the art and is described, for example, in Nimni, “Polypeptide Growth Factors: Targeted Delivery Systems,” Biomaterials, 10:1201-1225 (1997), incorporated herein by this reference, and articles referenced therein. In general, DBM is prepared from cortical bone of various animal sources. It is purified by a variety of procedures for the removal of non-collagenous proteins and other antigenic determinants. It typically consists of more than 99% Type I collagen. The DBM can be, for example, human DBM or rat DBM; DBM from other species can alternatively be used. For example, the DBM can be DBM from another animal such as a cow, a horse, a pig, a dog, a cat, a sheep, or another socially or economically important animal species.

DBM, which contains a mixture of bone morphogenic proteins (EMPs), consistently induces formation of new bone with a quantity of powdered matrices in the 10-25 mg range, while less than 10 mg fails to induce bone formation.

Accordingly, in attempts to produce better DBM, different processes have been investigated including those disclosed in (Muthukumaran et al., 1988, Col. Rel. Res., 8:433-441; Hammonds, et al., 1991, Mol. Endocrinol., 5:149-155; Ripamonti et al., 1992, Matrix, 12:202-212; Ripamonti et al., 1992, Plast. Reconstr. Surg., 89:731-739).

Generally, all of these methods produce DBM's which have the same inherent problems as the more traditional methods e.g. the products produced are comprised mainly of collagen, wherein the growth factors normally associated with bone are bound by binding proteins such that they are not readily available to patients' cells on administration. In recognition of this Urist and others have investigated the production and use of insoluble bone gelatin, which is a product produced by the further processing of DBM. Methods for isolating and purifying insoluble bone gelatin (ISBG) including, for example, in U.S. Pat. No. 4,294,753, as well as Urist et al. 1973, PNAS, 70; 12, pp 3511-3525 are well known. While Urist appreciated that DBM was not as capable of inducing bone formation as it should have been and that the lack of growth factors, especially BMP's was probably the cause, the methods disclosed by Urist had major flaws. For example, by treating DBM with chloroform and methanol many of the growth factors such as fibroblast growth factor (FGF) and transforming growth factor beta (TGF-β) were effectively removed. Moreover, the material produced by Urist was not sufficiently pliable to be used in most orthopaedic settings. Table 1 compares the preparation, physical and biochemical properties of DBM, ISBG and ABG.

TABLE 1

| | | ISBG Urist et al. 1973, | DBM Chen et al. U.S. Pat. No. | DBM Nimni, 1997, |
|---|---|---|---|---|
| | ABG | PNAS., 70; 12, pp 3511-3525 | 6,180,606 | Biomaterials 10: 1201-1225 |
| Preparation | Washed in saline | Washed in saline | Washed in saline | Chloroform-methanol |
| | | | | extraction of bone tissue. |
| | | | | Dried overnight |
| | Mechanical grinding | Mechanical cutting at RT | Mechanical grinding at | Mechanical grinding at RT |
| | at 4° C. to less than | to ~3000 micron size | 4° C. to various sizes | to 72-850 micron particles |
| | 1000 micron particle | | | |
| | Decalcified in HCl | Decalcified in HCl at | Decalcified in HCl at | Decalcified in HCl for |
| | for less than 24 h | least 24 h | least than 24 h | less than 24 h |
| | No chloroform- | Chloroform-methanol | No Chloroform-methanol | N/A |
| | methanol extraction | extraction of bone tissue | extraction used | |
| | Treated with | Treated with either LiCl | HCl only. Time | N/A |
| | EDTA/CaCl₂; less | (24 h)/EDTA (4 h)/CaCl₂ (24 h) | dependent on the amount | |
| | than 24 h in order | | of calcium required to | |
| | to enhance growth | | be removed | |
| | factor activity | | | |
| | Process at 4° C./below | Ambient to 4° C. | Ambient to 4° C. | Ambient to 4° C. |
| | N/A | N/A | Treated with chaotropic | N/A |
| | | | agent (& protease) | |
| Physical | Yellow-cream colour | White colour | White colour | White colour |
| properties | | | | |
| | Gelatin-like | Rubber-like | Powder | Powder |
| | Very soft | soft | soft | soft |
| Biochemical | BMP's, FGF, TGF-β, | BMP only | Only Type I collagen | 99% Type I collagen, |
| properties | IGF and growth | | | BMP's; growth factors |
| | factor binding | | | generally not active |
| | proteins eg IGF and | | | |
| | BMP binding protein | | | |
| | and any combination | | | |

In some embodiments of the present invention, ABG is prepared from milled bone powder up to about 1.0 millimetre particles (1000 microns). The powdered bone is pre-washed with saline at 35-55° C., preferably 40-45° C. for 5 minutes. This washing procedure replaced the chloroform and methanol solution as described by Urist. The washing with warm saline removed lipids and bone marrow cells in the tissue. Using this procedure, 800 of lipids and bone marrow cells were removed at the end of washing. The bone powder rinsed with saline is clear, moist and not overly dry as compared to bone powder treated with a solution of chloroform and methanol.

The milled bone powder is then demineralized using an acid such as hydrochloric acid or acetic acid, then treated with a neutralizing salt such as calcium chloride or calcium phosphate, and then treated with a stabilizer such as ethylene diamine tetraacetic acid (EDTA) all at 4° C. The resulting ABG is then treated with sterilized water. The entire procedure takes approximately 48 hours as it is desirable to reduce the amount of processing time in order to maximize the amount of liable growth factors retained in the ABG. It should be noted that no chloroform or methanol extraction is used in the process.

The following two protocols are particular useful in the present invention; however, it'will be appreciated by those skilled in the art that variations can be undertook without adversely affecting the ABG produced.

### Protocol 1

Bone powders prepared by the method described above were treated as follows:

Step 1 0.6 N HCl up to 24 hours at 4° C.;
Step 2 2.0 M CaCl₂ for 24 hours at 4° C.;
Step 3 0.5 M EDTA for 24 hours at 4° C.;
Step 4 8.0 M LiCl for 4 hours at 4° C.; and
Step 5 sterilized H₂O for 4 hours at 55° C.

### Protocol 2

Bone powders prepared by the method described above were treated as follows:

Step 1 0.6 N HCl up to 12 hours at 4° C.;
Step 2 2.0 M CaCl₂ up to 12 hours at 4° C.;
Step 3 0.5 M EDTA for 4 hours at 4° C.; and
Step 4 sterilized H₂O for 4 hours at 55° C.

To eliminate non-crucial chemicals, a series of experiments was conducted to examine if the use of solutions of chloroform and methanol, and lithium chloride (LiCl) are necessary for isolating and purifying ABG. Based on the results of rat models, it was found that neither a solution of chloroform and methanol, nor a solution of LiCl is essential to produce ABG that is suitable for induction of bone formation. By eliminating one or both of these chemicals from the isolation and purification procedure, the duration of ABG extraction is reduced by up to approximately one-half according to the present invention.

Once the ABG is produced it can be used directly to enhance bone formation or, in some aspects, the ABG is mixed with bone, forming cells ie seeded with a bone forming amount of bone-forming cells to produce a bone forming carrier material. The term “bone forming amount” refers to the volume or number of “bone-forming cells” that are seeded into the ABG so as to produce a therapeutically effective amount of bone tissue on implantation. For example, it is know that between about 1×10⁴ to about 1×10⁶ osteoblast cells per 0.01 g scaffold is a sufficient cell load to enable the cells to grow in vitro to completely populate the scaffold with cells with 2-3 weeks of culture.

It will be appreciated by those skilled in the art that any type of bone-forming cell might be used. Accordingly, the term “bone-forming cells” includes all cell types capable of growing new bone or leading to the growth of new bone. Cells known to be capable of growing new bone include, for example, osteoblasts, mesenchymal stem cells (MSCs), progenitor cells and combinations thereof. While bone-forming cells are generally found in bone marrow, as described below bone marrow per se requires purification before it can be used.

Bone marrow is a special, spongy, fatty tissue that houses stem cells, located inside a few large bones. These stem cells transform into white blood cells, red blood cells, platelets as well as specialised cells such as osteoblasts and osteoclasts.

Osteoblasts are cells which originate in the bone marrow and contribute to the production of new bone. Osteoblasts build up the matrix of the bone structure and also play a role in the mineralization of the bone matrix. Bone is constantly being built up and broken down by the body, making osteoblasts rather critical. The counterpart to the osteoblast is the osteoclast, a cell which is responsible for breaking down bone.

Osteoblasts and osteoclasts can further differentiate into several different cell types, in addition to working to build up bone. An osteocyte is an osteoblast which becomes trapped in the bone matrix. Osteocytes cannot divide, and they develop long extensions to communicate with other osteocytes. These cells move nutrients and waste through the bone matrix. Bone-lining cells are osteoblasts which become attached to the surface of the bone, flattening in the process.

Bone-lining cells appear to play a role in the activation of osteoblasts and osteoclasts, responding to hormones and changing conditions in the body to trigger the most appropriate activity. They also regulate the amount of calcium which can enter the bone, acting as a selectively permeable membrane which can either allow calcium to flow across into the bone, or block calcium absorption.

The osteoblasts are critical to the structure and integrity of the bone. They do not just build new bone, they also maintain and strengthen existing bone, ensuring that the matrix is not compromised and that it is as even as possible.

In some embodiments the bone-forming cells are a heterogeneous population of cells but do not include cells producing whole bone marrow. If bone marrow is used it is at least purified to remove red blood cells (erythrocytes).

Preferably the bone-forming cells are a purified or substantially purified population of cells. For example, after erythrocytes are removed from the bone marrow the resultant bone marrow cells may be cultured or otherwise separated to produce a purified or substantially purified population of cells. Methods for separating erythrocytes and specific cells from bone marrow are well known by persons skilled in the art and are also discussed infra.

The term “purified population” with reference to cells, as used herein, means a sample of cells composed of a single cell type. However, it would be appreciated by a person skilled in the art that a population of purified cells may contain very low numbers of other cells. Likewise, a “substantially purified population of cells” refers to a sample of cells composed predominately of a single cell type, but contains a minimal number of contaminating cells.

In some embodiments, the bone-forming cells are mesenchymal stem cells (MSCs) or progenitor cells. As used herein, the term MSCs refers to a cell capable of giving rise to differentiated cells in multiple mesenchymal lineages, specifically to osteoblasts, adipocytes, myoblasts and chondroblasts. Generally, mesenchymal stem cells also have one or more of the following properties: an ability to undergo asynchronous, or symmetric replication, that is where the two daughter cells after division can have different phenotypes; extensive self-renewal capacity; and clonal regeneration of the tissue in which they exist, for example, the non-hematopoietic cells of bone marrow. “Progenitor cells” differ from stem cells in that they typically do not have the extensive self-renewal capacity. In contrast to previously reported MSC and multipotent mesenchymal cell populations, the cells of the invention do not require lengthy time in culture prior to the appearance of the MSC phenotype, i.e. cells with the MSC phenotype and are responsive to canonical signaling pathways are present in freshly isolated or primary cultures that have been cultured for less than about 20 passages; usually less than about 10 passages.

MSC have been harvested from the supportive stroma of a variety of tissues. In both mouse and human a candidate population of cells has been identified in subcutaneous adipose tissue (AMSC). These cells have demonstrated the same in vitro differentiation capacity as BM-MSC for the mesenchymal lineages, osteoblasts, chondrocytes, myocytes, neurons, and adipocytes (Zuk et al. (2002) Mol Biol Cell 13, 4279-95; Fujimura et al. (2005) Biochem Biophys Res Commun 333, 116-21). Additionally, cell surface antigen profiling of these cells has revealed similar cell surface marker characteristics as the more widely studied BM-MSC (Simmons et al. (1994) Prog Clin Biol Res 389, 271-80; and Gronthos et al. (2001) J Cell Physiol 189, 54-63).

MSC may be characterized by both the presence of markers associated with specific epitopes identified by antibodies and the absence of certain markers as identified by the lack of binding of specific antibodies. MSC may also be identified by functional assays both in vitro and in vivo, particularly assays relating to the ability of stem cells to give rise to multiple differentiated progeny; assays for responsiveness to canonical signaling; and the like.

The bone forming cells are typically mammalian, where the term refers to any animal classified as a mammal, including humans, domestic and farm animals, and zoo, laboratory, sports, or pet animals, such as dogs, horses, cats, cows, mice, rats, rabbits, etc. Preferably, the mammal is human.

The bone-forming cells which are employed may be fresh, frozen, or have been subject to prior culture. They may be fetal, neonate, adult. The bone-forming cells may be initially obtained from adipose tissue (see U.S. Patent application 20030082152); bone marrow (Pittenger et al. (1999) Science 284(5411):143-147; Liechty et al. (2000) Nature Medicine 6:1282-1286); G-CSF or GM-CSF mobilized peripheral blood (Tondreau et al. (2005) Stem Cells 23(8): 1105-1112), or any other conventional source. However, it will be appreciated by those skilled in the art that bone-forming cells isolated from impure sources such as bone marrow will need to be at least partially purified to remove contaminating cells such as red blood cells before use. It will also be appreciated by those skilled in the art that the cells can be autologous.

In some embodiments, the bone forming cells are MSCs that have been maintained for at least about two passages; at least about five passages; at least about ten passages; or more in vitro. The MSC's may, in some circumstances, be cultured in osteogenic culture conditions before being mixed with the allogenic bone gel. Differentiating cells are obtained by culturing or differentiating MSC in a growth environment that enriches for cells with the desired phenotype, e.g. osteoblasts, osteogenic progenitor cells, etc. The culture may comprise agents that enhance differentiation to a specific lineage.

Osteogenic differentiation may be performed by plating cells and culturing to confluency, then culturing in medium comprising TGF-β1 and/or TGF-β3 at a concentration of from around about 100 pg/ml to around about 100 ng/ml, usually around about 10 ng/ml. Alternatively, osteogenic differentiation may be performed by incubating cells in a media supplemented with ascorbic acid, beta-glycerophosphate, and dexamethasone or BMP-2. Osteogenic cells may be identified by any number of means know to a person of skill in the art. For example, osteogenic cells may be identified by the expression of one or more osteoblast special genes including collagen type I, osteopontin and osteocalcin.

Following the differentiation in culture, the culture will usually comprise at least about 25% of the desired differentiated cells; more usually at least about 50% differentiated cells; at least about 75% differentiated cells, or more. The cells thus obtained may be used directly in the allogenic bone gel, or may be further isolated, e.g. in a negative selection to remove MSCs and other undifferentiated cells. Further enrichment for the desired cell type may be obtained by selection for markers characteristic of the cells, e.g. by flow cytometry, magnetic bead separation, panning, etc., as known in the art.

Once the desired bone-forming cells have been isolated and/or obtained they are then mixed with the allogenic bone gel to form the bone forming carrier material of the present invention, which carrier has anti-inflammation properties. The term “inflammation,” as used herein refers to an adverse immune response having a detrimental health effect in a subject. A “subject” is a vertebrate, preferably a mammal, more preferably a human. Mammals include, but are not limited to, humans, farm animals, sport animals, and pets.

It is well understood that inflammation is the first response of the immune system to infection or irritation and may be referred to as the innate cascade. Inflammation has two components: (i) cellular and (ii) exudative.

The exudative component involves the movement of fluid, usually containing many important proteins such as fibrin and immunoglobulins. Blood vessels are dilated upstream of an infection and constricted downstream while capillary permeability to the affected tissue is increased, resulting in a net loss of blood plasma into the tissue, giving rise to oedema or swelling.

The cellular component involves the movement of white blood cells from blood vessels into the inflamed tissue. The white blood cells, or leukocytes, take on a role in inflammation; they extravasate from the capillaries into tissue, and act as phagocytes, picking up bacteria and cellular debris. For instance, without being limited to any theory, lymphocytes and monocytes recruited to the inflamed tissue and also macrophages release chemokines that further recruit polymorphonuclear leukocytes. White blood cells may also aid by walling off an infection and preventing its spread.

Thus, the bone forming carrier material of the present invention is capable of modulating inflammatory cells including, but not limited to, monocytes, lymphocytes, eosinophils, neutrophils and basophils across the epithelial surface. Preferably, the inflammatory cells comprise neutrophils, such as polymorphonuclear leukocytes (“PMNs”). In particular, bone forming carrier material of the present invention is suitable for inhibiting the influx of polymorphonuclear leukocytes (PMNs) into a tissue involved in inflammation.

As used herein, the term “modulating” means regulating or controlling as necessary, through eliminating, reducing, maintaining or increasing a desired effect. The desired effect can be an effect on inflammatory cell migration or transmigration or by reducing the symptoms of inflammation as described supra. In particular, the bone forming carrier material reduces inflammation by reducing the number of PMNs in a tissue by at least 3 fold.

The activity of the bone forming carrier material to reduce inflammation can alternatively referred to as “anti-inflammatory” activity, a term which is intended to include inflammatory response modifier, including all inflammatory responses such as production of stress proteins, white blood cell infiltration, fever, pain, swelling and so forth.

In some embodiments, the bone forming carrier material of the present invention is used directly as described herein. In other embodiments, the bone forming carrier material is further formulated or manufactured into a material suitable for implantation and/or controlled-release of biologically active agents. The bone forming carrier material of the invention may be prepared by combining the bone forming carrier material with a selected supplementary material. The supplementary material is selected based upon its compatibility with the bone forming carrier material and the other components and its ability to impart properties (biological, chemical, physical, or mechanical) to the bone forming carrier material, which are desirable for a particular prophylactic or therapeutic purpose. For example, the supplementary material may be selected to improve tensile strength and hardness, increase fracture toughness, and provide imaging capability of the material after implantation. The supplementary materials are desirably biocompatible. The supplementary material may also be selected as a cohesiveness agent.

The supplementary material may be added to the bone forming carrier material in varying amounts and in a variety of physical forms, dependent upon the anticipated prophylactic or therapeutic use. For example, the supplementary material may be in the form of solid structures, such as sponges, meshes, films, fibres, gels, filaments or particles, including microparticles and nanoparticles. The supplementary material may be a composite, a particulate or liquid additive which is intimately mixed with the bone forming carrier material. For example, the supplementary material may be dissolved in a non-aqueous liquid prior to mixing with the bone forming carrier material.

In some embodiments, the supplementary material includes bone substitutes such as bone ceramics eg calcium phosphate ceramics including hydroxyapatites, tricalcium phosphate and biphasic calcium phosphate; calcium sulphate ceramics; and bioglass ie a group of artificial bone graft substitutes consisting of silico-phosphatic substitutes. In other embodiments, the supplementary material includes corals and porous coralline ceramics, including natural corals and synthetic porous coated hydroxyapatites.

In many instances, it is desirable that the supplementary material be bioresorbable. Bioresorbable material for use as supplementary material in the bone forming carrier material of the invention include, without limitation, polysaccharides, nucleic acids, carbohydrates, proteins, polypeptides, poly (α-hydroxy acids), poly(lactones), poly(amino acids), poly(anhydrides), poly(orthoesters), poly (anhydride-co-imides), poly(orthocarbonates), poly(α-hydroxy alkanoates), poly(dioxanones), and poly(phosphoesters). Preferably, the bioresorbable polymer is a naturally occurring polymer, such as collagen, glycogen, chitin, starch, keratins, silk, and hyaluronic acid; or a synthetic polymer, such as poly(L-lactide) (PLLA), poly(D,L-lactide) (PDLLA), polyglycolide (PGA), poly(lactide-co-glycolide (PLGA), poly(L-lactide-co-D, L-lactide), poly(D,L-lactide-co-trimethylene carbonate), polyhydroxybutyrate (PHB), poly(c-caprolactone), poly(δ-valerolactone), poly(γ-butyrolactone), poly(caprolactone), or copolymers thereof. Such polymers are known to bioerode and are suitable for use in the bone forming carrier material of the invention. In addition, bioresorbable inorganic supplementary materials, such as compositions including SiO₂, Na₂O, CaO, P₂O₅, Al₂O₃ and/or CaF₂, may be used, as well as salts, e.g., NaCl, and sugars, e.g., mannitol, and combinations thereof.

Supplementary materials may also be selected from non-resorbable or poorly resorbable materials. Suitable non-resorbable or poorly resorbable materials for use in the bone forming carrier material of the invention include, without limitation, dextrans, cellulose and derivatives thereof (e.g., methylcellulose, carboxy methylcellulose, hydroxypropyl methylcellulose, and hydroxyethyl cellulose), polyethylene, polymethylmethacrylate (PMMA), carbon fibers, poly(ethylene glycol), poly(ethylene oxide), polyvinyl alcohol), poly(vinylpyrrolidone), poly(ethyloxazoline), poly(ethylene oxide)-co-poly(propylene oxide) block copolymers, poly(ethylene terephthalate)polyamide, and lubricants, such as polymer waxes, lipids and fatty acids.

The bone forming carrier material of the invention is useful for the controlled-release of biologically active agents. In general, the only requirement is that the substance is encased within the material and remain active within the implantable material during fabrication or be capable of being subsequently activated or re-activated, or that the biologically active agent can be added at the time of implantation of the bone forming carrier material into a subject.

Biologically active agents that can be incorporated into the bone forming carrier material of the invention include, without limitation, organic molecules, inorganic materials, proteins, peptides, nucleic acids (e.g., genes, gene fragments, gene regulatory sequences, and antisense molecules), nucleoproteins, polysaccharides, glycoproteins, and lipoproteins. Classes of biologically active compounds that can be loaded into a implantable material of the invention include, without limitation, anti-cancer agents, antibiotics, analgesics, anti-inflammatory agents, immunosuppressants, enzyme inhibitors, antihistamines, anti-convulsants, hormones, muscle relaxants, anti-spasmodics, prostaglandins, anti-depressants, anti-psychotic substances, trophic factors, osteoinductive proteins, growth factors, and vaccines.

Anti-cancer agents include alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF α agonists/antagonists, endothelin A receptor antagonists, retinoic acid receptor agonists, immuno-modulators, hormonal and antihormonal agents, photodynamic agents, and tyrosine kinase inhibitors.

Antibiotics include aminoglycosides (e.g., gentamicin, tobramycin, netilmicin, streptomycin, amikacin, neomycin), bacitracin, corbapenems (e.g., imipenem/cislastatin), cephalosporins, colistin, methenamine, monobactams (e.g., aztreonam), penicillins (e.g., penicillin G, penicillin V, methicillin, natcillin, oxacillin, cloxacillin, dicloxacillin, ampicillin, amoxicillin, carbenicillin, ticarcillin, piperacillin, mezlocillin, azlocillin), polymyxin B, quinolones, and vancomycin; and bacteriostatic agents such as chloramphenicol, clindanyan, macrolides (e.g., erythromycin, azithromycin, clarithromycin), lincomyan, nitrofurantoin, sulfonamides, tetracyclines (e.g., tetracycline, doxycycline, minocycline, demeclocyline), and trimethoprim. Also included are metronidazole, fluoroquinolones, and ritampin.

Enzyme inhibitors are substances which inhibit an enzymatic reaction. Examples of enzyme inhibitors include edrophonium chloride, N-methylphysostigmine, neostigmine bromide, physostigmine sulfate, tacrine, tacrine, 1-hydroxy maleate, iodotubercidin, p-bromotetramisole, 10-(alpha-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylamine, N₆-monomethyl-L-arginine acetate, carbidopa, 3-hydroxybenzylhydrazine, hydralazine, clorgyline, deprenyl, hydroxylamine, iproniazid phosphate, G-MeO-tetrahydro-9H-pyrido-indole, nialamide, pargyline, quinacrine, semicarbazide, tranylcypromine, N,N-diethylaminoethyl-2,2-diphenylvalerate hydrochloride, 3-isobutyl-1-methylxanthne, papaverine, indomethacind, 2-cyclooctyl-2-hydroxyethylamine hydrochloride, 2,3-dichloro-a-methylbenzylamine (DCMB), 8,9-dichloro-2,3,4,5-tetrahydro-1H-2-benzazepine hydrochloride, p-aminoglutethimide, p-aminoglutethimide tartrate, 3-iodotyrosine, alpha-methyltyrosine, acetazolamide, dichlorphenamide, 6-hydroxy-2-benzothiazolesulfonamide, and allopurinol.

Antihistamines include pyrilamine, chlorpheniramine, and tetrahydrazoline, among others.

Anti-inflammatory agents include corticosteroids, non-steroidal anti-inflammatory drugs (e.g., aspirin, phenylbutazone, indomethacin, sulindac, tolmetin, ibuprofen, piroxicam, and fenamates), acetaminophen, phenacetin, gold salts, chloroquine, D-Penicillamine, methotrexate colchicine, allopurinol, probenecid, and sulfinpyrazone.

Muscle relaxants include mephenesin, methocarbomal, cyclobenzaprine hydrochloride, trihexylphenidyl hydrochloride, levodopa/carbidopa, and biperiden.

Anti-spasmodics include atropine, scopolamine, oxyphenonium, and papaverine.

Analgesics include aspirin, phenybutazone, idomethacin, sulindac, tolmetic, ibuprofen, piroxicam, fenamates, acetaminophen, phenacetin, morphine sulfate, codeine sulfate, meperidine, nalorphine, opioids (e.g., codeine sulfate, fentanyl citrate, hydrocodone bitartrate, loperamide, morphine sulfate, noscapine, norcodeine, normorphine, thebaine, nor-binaltorphimine, buprenorphine, chlomaltrexamine, funaltrexamione, nalbuphine, nalorphine, naloxone, naloxonazine, naltrexone, and naltrindole), procaine, lidocain, tetracaine and dibucaine.

Prostaglandins are art recognized and are a class of naturally occurring chemically related, long-chain hydroxy fatty acids that have a variety of biological effects.

Anti-depressants are substances capable of preventing or relieving depression. Examples of anti-depressants include imipramine, amitriptyline, nortriptyline, protriptyline, desipramine, amoxapine, doxepin, maprotiline, tranylcypromine, phenelzine, and isocarboxazide.

Trophic factors are factors whose continued presence improves the viability or longevity of a cell. Trophic factors include, without limitation, platelet-derived growth factor (PDGP), neutrophil-activating protein, monocyte chemoattractant protein, macrophage-inflammatory protein, platelet factor, platelet basic protein, and melanoma growth stimulating activity; epidermal growth factor, transforming growth factor (alpha), fibroblast growth factor, platelet-derived endothelial cell growth factor, insulin-like growth factor, glial derived growth neurotrophic factor, ciliary neurotrophic factor, nerve growth factor, bone growth/cartilage-inducing factor (alpha and beta), bone morphogenetic proteins, interleukins (e.g., interleukin inhibitors or interleukin receptors, including interleukin 1 through interleukin 10), interferons (e.g., interferon alpha, beta and gamma), hematopoietic factors, including erythropoietin, granulocyte colony stimulating factor, macrophage colony stimulating factor and granulocyte-macrophage colony stimulating factor; tumor necrosis factors, and transforming growth factors (beta), including beta-1, beta-2, beta-3, inhibin, and activin.

Hormones include estrogens (e.g., estradiol, estrone, estriol, diethylstibestrol, quinestrol, chlorotrianisene, ethinyl estradiol, mestranol), anti-estrogens (e.g., clomiphene, tamoxifen), progestins (e.g., medroxyprogesterone, norethindrone, hydroxyprogesterone, norgestrel), antiprogestin (mifepristone), androgens (e.g, testosterone cypionate, fluoxymesterone, danazol, testolactone), anti-androgens (e.g., cyproterone acetate, flutamide), thyroid hormones (e.g., triiodothyronne, thyroxine, propylthiouracil, methimazole, and iodixode), and pituitary hormones (e.g., corticotropin, sumutotropin, oxytocin, and vasopressin).

The biologically active agent is desirably selected from the family of proteins known as the transforming growth factors-beta (TGF-β) superfamily of proteins, which includes the activins, inhibins and bone morphogenetic proteins (BMPs). Most preferably, the active agent includes at least one protein selected from the subclass of proteins known generally as BMPs, which have been disclosed to have osteogenic activity, and other growth and differentiation type activities. These BMPs include BMP proteins BMP-2, BMP-3, BMP-4, BMP-5, BMP-6 and BMP-7 (OP-1), disclosed for instance in U.S. Pat. Nos. 5,108,922; 5,013,649; 5,116,738; 5,106,748; 5,187,076; and 5,141,905; BMP-8, disclosed in PCT publication WO91/18098; and BMP-9, disclosed in PCT publication WO93/00432, BMP-10, disclosed in PCT application WO94/26893; BMP-11, disclosed in PCT application WO94/26892, or BMP-12 or BMP-13, disclosed in PCT application WO 95/16035; BMP-14; BMP-15, disclosed in U.S. Pat. No. 5,635,372; or BMP-16, disclosed in U.S. Pat. No. 5,965,403. Other TGF-.beta. proteins which may be useful as the active agent in the bone forming carrier material of the invention include Vgr-2, Jones et al., Mol. Endocrinol. 6:1961 (1992), and any of the growth and differentiation factors (GDFs), including those described in PCT applications WO94/15965; WO94/15949; WO95/01801; WO95/01802; WO94/21681; WO94/15966; WO95/10539; WO96/01845; WO96/02559 and others.

Also useful in the invention may be BIP, disclosed in WO94/01557; HP00269, disclosed in JP Publication number: 7-250688; and BMP-14 (also known as MP52, CDMP1, and GDF5), disclosed in PCT application WO93/16099. The disclosures of all of the above applications are incorporated herein by reference. A subset of BMPs which are presently preferred for use in the invention include BMP-2, BMP-3, BMP-3b, BMP-4, BMP-5, BMP-6, BMP-7, BMP-8, BMP-9, BMP-10, BMP-11, BMP-12, BMP-13, BMP-14, BMP-15, BMP-16, BMP-17, and BMP-18. The active agent is most preferably BMP-2, the sequence of which is disclosed in U.S. Pat. No. 5,013,649, the disclosure of which is incorporated herein by reference. Other osteogenic agents known in the art can also be used, such as teriparatide (Forteo™), Chrysalin™, prostaglandin E2, or LIM protein, among others.

The biologically active agent may be recombinantly produced, or purified from a protein composition. The active agent, if a TGF-β such as a BMP, or other dimeric protein, may be homodimeric, or may be heterodimeric with other BMPs (e.g., a heterodimer composed of one monomer each of BMP-2 and BMP-6) or with other members of the TGF-β superfamily, such as activins, inhibins and TGF-β1 (e.g., a heterodimer composed of one monomer each of a BMP and a related member of the TGF-β superfamily). Examples of such heterodimeric proteins are described for example in Published PCT Patent Application WO 93/09229, the specification of which is hereby incorporated herein by reference.

The amount of osteogenic protein effective to stimulate increased osteogenic activity of present or infiltrating progenitor or other cells will depend upon the size and nature of the defect being treated. Generally, the amount of protein to be delivered is in a range of from about 0.1 to about 100 mg; preferably about 1 to about 100 mg; most preferably about 10 to about 80 mg.

Biologically active agents can be introduced into the bone forming carrier material of the invention during or after its formation. Agents may conveniently be mixed into the bone forming carrier material.

Standard protocols and regimens for delivery of the above-listed agents are known in the art. Typically, these protocols are based on oral or intravenous delivery. Biologically active agents are introduced into the implantable material in amounts that allow delivery of an appropriate dosage of the agent to the implant site. In most cases, dosages are determined using guidelines known to practitioners and applicable to the particular agent in question. The exemplary amount of biologically active agent to be included in the bone forming carrier material of the invention is likely to depend on such variables as the type and extent of the condition, the overall health status of the particular patient, the formulation of the active agent, and the bioresorbability of the implantable material used. Standard clinical trials may be used to optimize the dose and dosing frequency for any particular biologically active agent.

The bone forming carrier material of the invention can be used to deliver biologically active agents to any of a variety of sites in a mammalian body, preferably in a human body. The bone forming carrier material can be implanted subcutaneously, intramuscularly, intraperitoneally and bony sites. Preferably, the bone forming carrier material is implanted into or adjacent to the tissue to be treated such that, by diffusion, the encased biologically active agent is capable of penetrating the tissue to be treated.

Such materials offer the advantage of controlled, localized delivery. As a result, less biologically active agent is required to achieve a therapeutic result in comparison to systemic administration, reducing the potential for side effects maximizing the agent's activity at the site of implantation.

The bone forming carrier material can be implanted into any acceptable tissue. The bone forming carrier material has particular advantages for delivery of biologically active agents to sites in bone. Implantation of the bone forming carrier material to a bony site includes either anchoring the vehicle to a bone or to a site adjacent to the bone.

The bone forming carrier material described herein can be implanted to support bone growth so that it is eventually replaced by the subject's own bone. It should be borne in mind, however, that bone ingrowth may well affect the resorbability rate of the drug delivery for implantable material incorporating a biologically active agent. Accordingly, it may be desirable in certain circumstances (e.g., where the biologically active agent must be delivered according to a precise, predetermined administrative schedule) to reduce bone growth into the drug delivery vehicle, for example by blocking penetration of osteocytic or chondrocytic cells or precursors. In most circumstances, ossification can be avoided by placing the device at some distance away from bone. Generally, 1 mm will be sufficient, although greater distances are preferred.

Other steps may also be taken to augment ossification, including introduction bone forming cells harvested from the patient into the graft, or incorporation of trophic factors or bone growth inducing proteins into, or onto the device. Non-autologous bone cells can also be used to promote bone regeneration. Immunosuppressants may be administered to the device recipient, either systemically or by incorporation into the device. Thus, cells or tissues obtained from primary sources, cell lines or cell banks may be used (See, U.S. Pat. No. 6,132,463 to Lee et al., which is incorporated herein by reference).

Certain categories of biologically active agents are expected to be particularly suitable for delivery to bony sites. For example, where the bone forming carrier material is applied to a damaged bone site, it may be desirable to incorporate bone regenerative proteins (BRPs) into the bone forming carrier material. BRPs have been demonstrated to increase the rate of bone growth and to accelerate bone healing (see, for example, Appel et al., Exp. Opin. Ther. Patents 4:1461 (1994)). Exemplary BRPs include, but are in no way limited to, Transforming Growth Factor-Beta (TGF-β), Cell-Attachment Factors (CAFs), Endothelial Growth Factors (EGFs), OP-1, and Bone Morphogenetic Proteins (BMPs). Such BRPs are currently being developed by Genetics Institute, Cambridge, Mass.; Genentech, Palo Alto, Calif.; and Creative Biomolecules, Hopkinton, Mass. Bone regenerative proteins and trophic factors can also be used to stimulate ectopic bone formation if desired. For example, an implantable material containing BMP-2 can be placed subcutaneously, and bone formation will occur within 2-4 weeks.

Antibiotics and antiseptics are also desirably delivered to bony sites using the bone forming carrier material of the invention. For example, as indicated supra one of the major clinical implications arising from bone-graft surgery is a need to control the post-operative inflammation or infection, particularly infection associated with osteomyelitis. The bone forming carrier material of the invention that includes an antibiotic can be used as, or in conjunction with, an improved bone graft to reduce the chances of local infection at the surgery site, contributing to infection-free, thus faster, bone healing process. The efficacy of antibiotics is further enhanced by controlling the resorption of the poorly crystalline hydroxyapatite such that it dissolves at a rate that delivers antibiotic peptides or its active component at the most effective dosage to the tissue repair site. Antibiotics and bone regenerating proteins may be incorporated together into the bone forming carrier material of the invention, to locally deliver most or all of the components necessary to facilitate optimum conditions for bone tissue repair.

Other biologically active agents that are desirably delivered to bony sites include anti-cancer agents, for example for treatment of bone tumors (see, for example, Otsuka et al., J. Pharm. Sci. 84:733 (1995)). The bone forming carrier material of the invention is also useful, for example, where a patient has had a bone tumor surgically removed, because the bone forming carrier material can be implanted to improve the mechanical integrity of the bone site while also treating any remaining cancer cells to avoid metastasis

Additional biologically active agents can be incorporated into the bone forming carrier material of the invention for delivery to bony sites include agents that relieve osteoporosis. For example, amidated salmon calcitonin has been demonstrated to be effective against osteoporosis.

Vitamin D and Vitamin K are also desirably delivered to bony sites, as are angiogenic factors such as VEGF, which can be used when it is desirable to increase vascularization.

The bone forming carrier material of the invention can be useful for treating or repairing a variety of orthopaedic conditions. The term “orthopaedic condition” means an condition associated with bones, connective tissue or the like. As such the present invention relates to the treatment of fractures in bones, defects in bone and the like or to immobilize joints during fusion procedures. The invention may also be used with prosthetic joints. In other embodiments the invention can be used to treat, repair or regenerate other tissues, including but not limited to connective tissue and cartilage and bone. Connective tissue can by treated as described in U.S. Pat. Nos. 5,197,985; 5,226,914 and 5,811,094 to Caplan et al., hereby incorporated herein by reference in their entirety. Chondrogenesis can be promoted as described in U.S. Pat. No. 5,908,784 to Johnstone et al., hereby incorporated herein by reference in its entirety.

“Treat” as used herein refers to any type of treatment or prevention that imparts a benefit to a subject afflicted with or at risk of developing an orthopaedic condition, including improvement in the condition of the subject (e.g., in one or more symptoms), delay in the progression of the orthopaedic condition, delay the onset of symptoms or slow the progression of symptoms, etc. As such, the term “treatment” also includes prophylactic treatment of the subject to prevent the onset of symptoms. As used herein, “treatment” and “prevention” are not necessarily meant to imply cure or complete abolition of symptoms, but refer to any type of treatment that imparts a benefit to a patient afflicted with an orthopaedic condition, including improvement in the condition of the patient (e.g., in one or more symptoms), delay in the progression of the orthopaedic condition, etc.

“Treatment effective amount”, “amount effective to treat” or the like as used herein means an amount of the bone forming carrier material sufficient to produce a desirable effect upon a patient inflicted with orthopaedic condition. This includes improvement in the condition of the patient (e.g., in one or more symptoms), delay in the progression of the orthopaedic condition, etc.

The bone forming carrier material of the invention may be implanted into the vertebral body for treatment of spinal fusion, spinal fractures, implanted into long bone or flat bone fractures to augment the fracture repair or to stabilize the fractured fragments, or implanted into intact osteoporotic bones to improve bone strength. It can be useful in the augmentation of a bone-screw or bone-implant interface. Additionally, it can be useful as bone filler in areas of the skeleton where bone may be deficient. Examples of situations where such deficiencies may exist include post-trauma with segmental bone loss, post-bone tumor surgery where bone has been excised, and after total joint arthroplasty. The bone forming carrier material can be used to hold and fix artificial joint components in subjects undergoing joint arthroplasty, as a strut to stabilize the anterior column of the spine after excision surgery, as a structural support for segmented bone (e.g., to assemble bone segments and support screws, external plates, and related internal fixation hardware), and as a bone graft substitute in spinal fusions.

The bone forming carrier material can be used per se or the bone forming carrier material can be used to coat medical devices such as prosthetic bone implants. For example, where the prosthetic bone implant has a porous surface, the bone forming carrier material may be applied to the surface to reduce inflammation and/or promote bone growth therein (i.e., bone ingrowth). The bone forming carrier material may also be applied to a prosthetic bone implant to enhance fixation within the bone.

The bone forming carrier material of the present invention are easy to apply and can be readily modeled to accurately reconstruct bony cavities, missing bone, and to recreate contour defects in skeletal bone. The bone forming carrier material can be applied, for example, with a spatula, can be moulded and sculpted, and can hold its shape satisfactorily until set.

By “comprising” is meant including, but not limited to, whatever follows the word comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The invention will now be further described by way of reference only to the following non-limiting examples. It should be understood, however, that the examples following are illustrative only, and should not be taken in any way as a restriction on the generality of the invention described above.

### Example 1

### Production of Allogenic Bone Gel

Allogenic bone gel was produced from up to 10 grams of milled bone, which was immersed in 36% HCl solution at 4° C. for 12 hours. The DBM was then immersed in 1000 ml 2.0 M CaCl₂ at 4° C. for 12 hours. After this, the material was immersed in 1000 ml 0.5 M EDTA for 4 hours at 4° C., and at the same time NaOH was added to the solution until pH 8.0 was reached. The resulting material was immersed into H₂O at 55° C. for 4 hours to produce the allogenic bone gel (ABG).

The ABG was then used directly as outline below or mixed with biologically active agents if required. For example, 0.5 g ABG was mixed with 0.5 g OP-1 (which contains 1.75 mg of recombinant human osteogenic protein 1 in 0.5 g of bovine collagen) to produce an implantable material that comprised at least 50% ABG. The recombinant human osteogenic protein 1 (OP-1) was provided by Stryker Biotech.

### Example 2

### Implantation of ABG in Animals

Thirty-two skeletally mature New Zealand White rabbits (age, 1 years old; weight, 3.5-4.5 kg) were divided randomly into four groups, and in each rabbit one of the following materials was implanted:

1). 1 g corticocancellous bone harvested from each side of the posterior iliac crest;
2). 1 g of ABG as produced in Example 1 was placed into each side of fusion bed;
3). 3.5 mg of recombinant human OP-1 in 1.0 g of bovine collagen for each side; and
4). ABG mixed with recombinant human OP-1 as described in Example 1.

Animals were housed in an established animal facility for a period of 1 week before surgery to allow acclimatization. Preoperative radiographs were obtained to rule out underlying disease.

Surgical anaesthesia was achieved with intramuscular injection of acepromazine (0.75 mg/kg) followed by ketamine (35 mg/kg) and xylazine (5 mg/kg) (Lipman et al., 1990) Enrofloxacin (5-10 mg/kg) was administered subcutaneously immediately before surgery.

The rabbits were shaved, positioned, draped, sterilised, and prepared in a standard surgical fashion. A dorsal midline skin incision is made in the lumbar region extending from L4-L7. Fascial incisions were made 2-3 cm on each side of the midline and a plane between the multifidus and longissimus muscles was made through blunt dissection until the transverse processes of L5-L6 and the intertransverse membrane exposed. Identification of vertebral levels was made by manual palpation of superficial landmarks using the sacrum as reference. The dorsal aspects of L5-L6 transverse processes were decorticated using a high-speed burr. Graft materials were then placed in the paraspinal muscle bed between the transverse processes. The wounds were closed using 3-0 absorbable sutures continuously to both the fascial and skin layers. Post-operative radiographs were taken to confirm the level of fusion.

All animals received 0.1 mg/kg buprenorphine for post-operative analgesia and were individually housed. There were no post-operative restrictions on activity, and no supportive orthotic devices were used.

Follow-up was 6 weeks post-operatively as fusions have been shown to be distinguishable from non-unions by this time in previous research (Boden et al., 1995, Spine, 20(4): 412-20; Minamide et al., 1999, Spine, 24(18): 1863-70; Namikawa et al., 2005, Spine, 30(15): 1717-22). Rabbits were killed with a sedating dose of intramuscular injection of xylazine (2.5 mg/kg) followed by a lethal dose of intravenous pentobarbital.

Fusion masses were characterized and compared with manual, radiographic, biomechanical, and histologic evaluations.

At the time of harvest, the operated segments and the rest of the lumbar spine were manually palpated to assess structural integrity by 2 blinded independent observers. Each segment was graded as solid or not solid. Only levels graded solid were considered fused.

All lumbar spines were examined by posteroanterior plain radiographs, mammography, and micro computed tomographic (microCT) scans (GE) 6 weeks post surgery. Each radiograph was assigned a numerical score using of the grading scale (see Table 2) by three observers in a blinded fashion (Yee et al., 2003, Spine, 28(21): 2435-40). Table 2 shows the radiographic grading of spine fusions.

TABLE 2

| Roentgenographic | |
|---|---|
| Score | Criteria |
| 4 | Intertransverse bone mass present |
| | bilaterally without lucency |
| 3 | Bone mass present bilaterally with |
| | lucency on one side only |
| 2 | Bone mass present bilaterally with |
| | lucency bilaterally |
| 1 | Bone mass present on one side only |
| 0 | No bone mass seen on either side |

Biomechanical testing to evaluate the strength of the L5-L6 fusion site was performed by three-point flexion-bending test using a materials testing machine.

Harvested specimens were fixed in 4% formalin in a neutral buffer solution, decalcified in 10% formic acid solution, dehydrated in a gradient ethanol series, and embedded in paraffin. Sections of 4 μm thickness at the intertransverse process region were cut in a sagittal plane, stained with hematoxylin and eosin, and observed under light microscopy to examine for the presence of bony fusion between the newly formed bone and transverse processes.

Average values were presented as mean±standard deviation. Fusion rates determined by manual palpation and radiographic analysis were evaluated using Fisher's exact test. Comparisons of biomechanical testing of spines in each group were made using one-way analysis of variance (ANOVA). Significance for all tests was defined as P<0.05.

Three rabbits were excluded (9%): one from autograft group died because of anaesthesia-related complications. Another two, one each from the OP-1 and ABG+OP-1 groups were sacrificed as they encountered deep wound infection. The remaining rabbits tolerated the surgical procedure without complications and started to gain weight after 1 week of post-operation.

Inspection by manual palpation of the fusion mass in the BMP group showed a bony mass in the intertransverse area that was more prominent than in the other groups. Solid spinal fusion was achieved in all seven rabbits in the ABG+OP-1 group (see Table 3).

TABLE 3
UNION RATE ON MANUAL PALPATION AFTER 6 WEEKS

| Group Material | Results |
|---|---|
| Autograft group | 3/7* |
| ABG group | 2/8** |
| OP-1 group | 2/7** |
| ABG + OP-1 group | 7/7*, ** |

Note:
Significant difference (Fisher's exact test, **P < 0.01 *P < 0.05).
PO = postoperative.

Six weeks after surgery, the degree of radiographic intertransverse processes fusion rate as assessed by the 5-point grading scale is presented in Table 4.

TABLE 4
RADIOGRAPHIC SCORE AFTER 6 WEEKS

| Group Material | Mean Value | Significance* |
|---|---|---|
| Autograft | 2.43 ± .98 | A/IO: P = 0.031 |
| group(A) | | |
| ABG group(I) | 2.12 ± .64 | I/IO: P = 0.004 |
| OP-1 group(O) | 2.29 ± .18 | O/IO: P = 0.014 |
| ABG + OP-1 | 3.71 ± .18 | |
| group(IO) | | |

Note:
The P values were derived using the One-way ANOVA, Bonferroni post hoc test.
A/IO = comparison between trial group A and IO;
I/IO = comparison between trial group I and IO;
O/IO = comparison between trial group O and IO

It can be seen that the ABG alone or combined with OP-1 produced equivalent or superior results as compared to the autogenous bone graft. The test was conducted in a critical bone defect model wherein the successful outcome was a solid posterolateral intertransverse process fusion. In the current study, autograft group did not result in a significant difference in fusion rate compared with results in the previous study in the same model (57% VS 66%) confirming the consistency of the model (Boden et al., J Bone Joint Surg Am., 77(9): 1404-17; Boden et al. (1995), Spine, 20(24): 2626-32).

Result of radiographic and histologic studies consistently showed fusion mass size of ABG/OP-1 composite group is larger than autograft, ABG alone and OP-1 alone. More mature fusion masses were also noted, ABG combined with OP-1 showed the greatest response in osteoid and new bone growth. However ABG alone and OP-1 alone showed osteoid formation, but no bony fusion after 6 weeks. Autograft showed more new bone growth than ABG alone and OP-1 alone. Quantitative Micro CT ray Tomography (MicroCT) results showed that bone volume in ABG/OP-1 group is significantly larger than the other three groups. We also found that the bone volume formed in outside zone is larger than central zone.

Pain relief and stability are the primary goals of spinal fusion. Although radiography and histology revealed fusion masses, these techniques can not be used to evaluate the stability of the fusion. Physiology biomechanical flexibility testing offers a precise method to characterize the changes in physiologic motion that result from spinal fusion. In the current study posterolateral fusion led to significant ROM decreases in lateral bending, flexion and extension between the ABG/OP-1 group and the autograft group.

| | Autograft | ABG | OP-1 | ABG + OP-1 |
|---|---|---|---|---|
| Osteogenic cell | + | ± | ± | ± |
| Osteoinductivity | + | + | ++ | +++ |
| Osteoconductivity | + | + | ± | + |

Of most interest was the observation that the ABG and/or ABG plus OP-1 group had reduced inflammation relative to the other groups.

### Example 3

### Anti-Inflammatory Properties of ABG

Inflammatory reaction caused by failure of arthroplasty, bacterial infection or tumour metastasis is a major concern in patients exhibiting osteolysis. Pro-inflammatory cytokines, such as IL-1, IL-6, TNF and the cascade reaction of bone inductive growth factors including OP-1 and BMP-2, are considered to be major mediators of osteolysis and ultimately aseptic loosening. In addition, lipopolysaccharide (LPS)-induced pro-inflammatory cytokine released in bone cells is also linked to bacterial bone infection.

Based on the previous observation that ABG could significantly reduce the inflammation caused by OP-1 or its carrier in rabbit model of spinal fusion (Example 2), we proposed that ABG may inhibit the inflammatory reaction caused by failure of arthroplasty, bacterial infection or tumour metastasis.

The LPS-induced osteolysis in the mouse calvarium model was used to examine the anti-inflammatory effect of ABG vivo. LPS with or without ABG was introduced into mouse calvaria. The method used is described by Yip et al. 2004, J Bone Miner Res., 19(11):1905-16 herein incorporated in its entirety by reference.

ABG was produced as described in Example 1. LPS (Escherichia coli, serotype 026-B6) (Sigma, Castle Hill, New South Wales, Australia) and Lycoll™ (Resorba, Nuernberg, Germany) were obtained through commercial outlets.

Twenty C57 Black mice were divided into four groups: sham operation+saline injection; sham operation+LPS injection; ABG implantation+LPS injection; and Lycoll™ implantation+LPS injection.

In the sham operation+saline injection group, a skin incision of 0.5 cm long was made on top of calvaria and an injection of saline (50 ml/mice) was given 3 days later. The sham operation+LPS group underwent the same operation procedure and were then given an injection of LPS (500 mg/mice) 3 days later. In the ABG implantation+LPS group, the same operation procedure was employed and about 0.1 g ABG was implanted into the space between the skin and the skull. Three days later, 500 mg LPS was injected into the same area for each mouse. For Lycoll™ implantation+LPS group the same procedures as in the previous group were used except Lycoll™ was implanted. After 7 days of injections, histo-pathological assessment was performed and micrographs taken at 100×.

Four days after the operation procedure, it was noticed that the skin around the injection area of 2 mice in sham operation+LPS group was significantly inflamed and warm to the touch. The eye on the same side of one of these 2 mice was swollen. This situation remained unchanged to the end of experimental period.

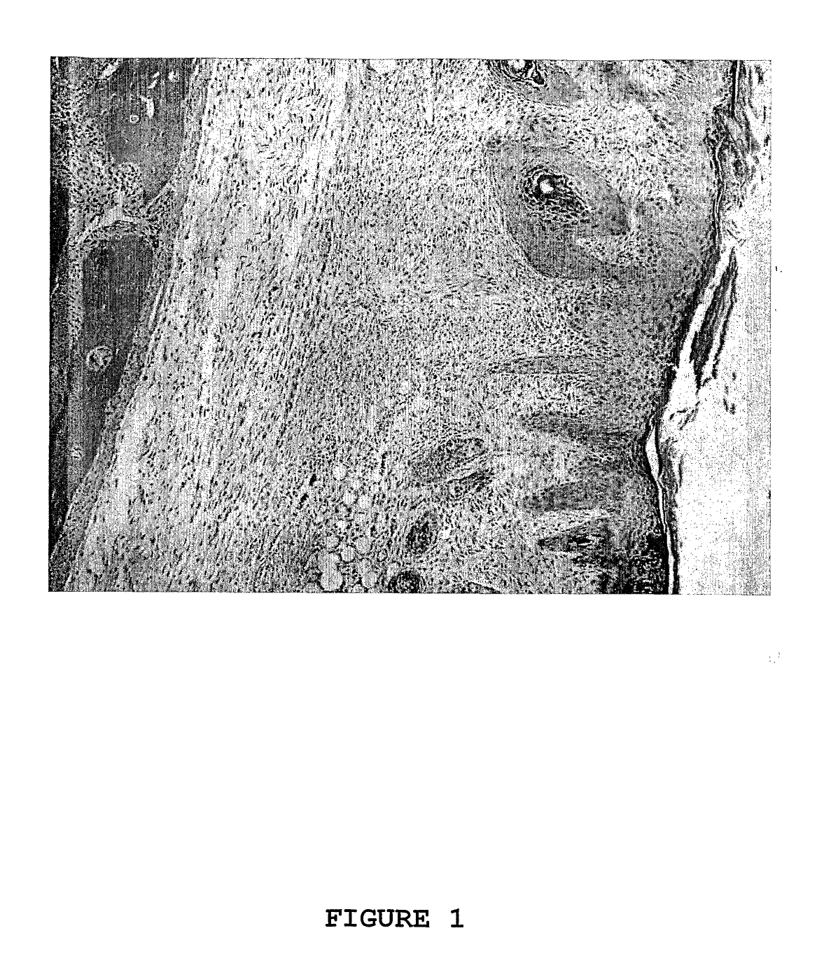

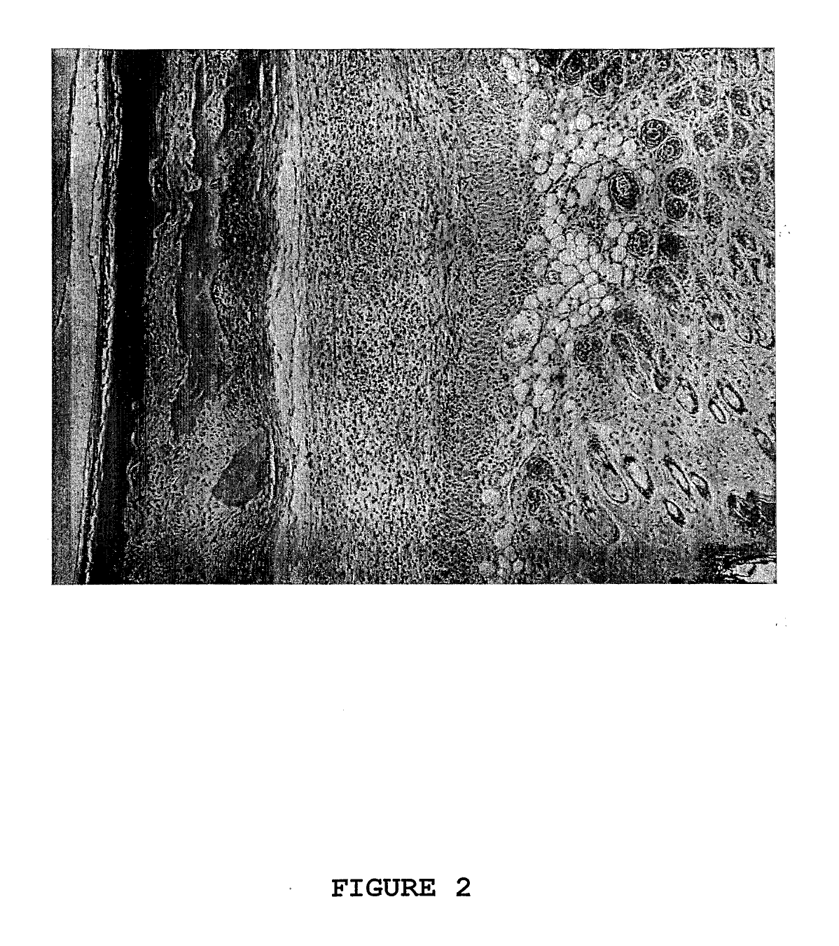

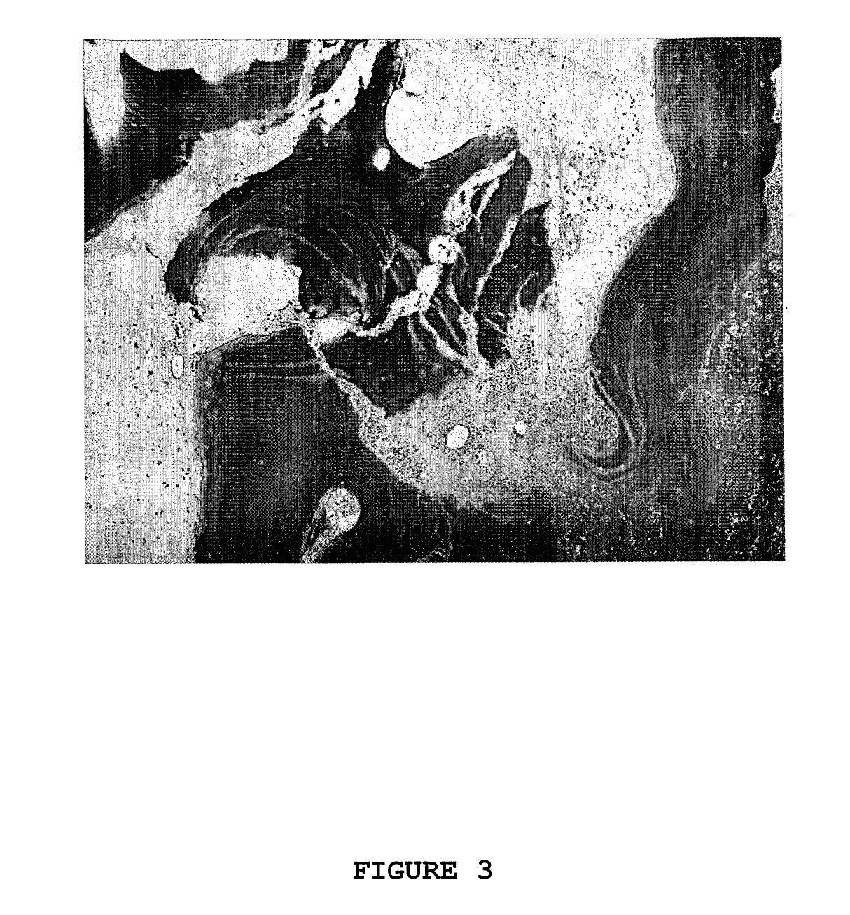

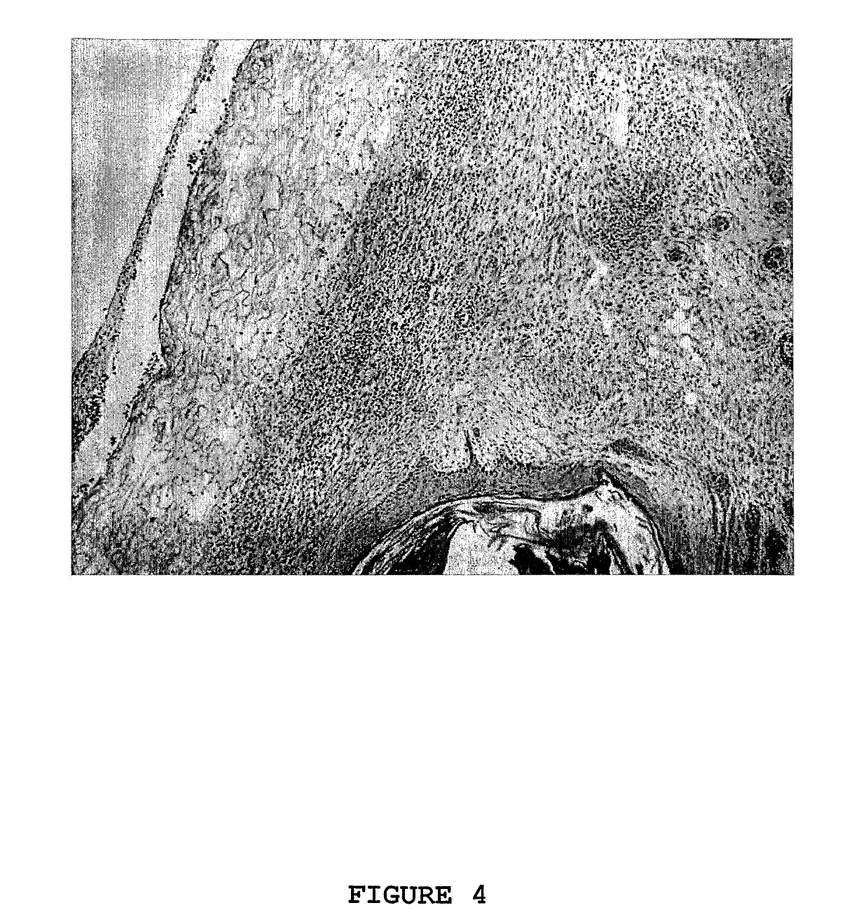

Histo-pathological samples from sham operation+saline injection group showed there were some acute inflammatory cells and fibroblasts in the injection area (FIG. 1), while the samples from sham operation+LPS injection group showed prominent vasodilatation of precapillary arterioles and densely packed polymorphonuclear leukocytes in connective tissue in the injection area (FIG. 2). In contrast, there were only a few polymorphonuclear leukocytes present in the operation area (FIG. 3) of samples from the ABG+LPS injection group. There was no notable difference found between the sham operation+LPS and Lycoll™+LPS (FIG. 4) group,—in terms of inflammatory reaction.

This experiment demonstrated that ABG inhibits LPS-induced inflammation in mouse model. Combining these results with our observation that ABG could significantly reduce the inflammation caused by OP-1 in a rabbit model of spinal fusion (Example 2), we conclude that ABG can inhibit the inflammatory reaction caused by the failure of arthroplasty, bacterial infection or tumour metastasis; the inflammatory reaction caused by other implant materials or carriers in the administration of OP-1, BMP-2 or other biologically active agents.

### Example 4

### Evaluation of Allogenic Bone Gel as a Carrier for Autologous Bone-Forming Cells in Vitro

We have previously shown that allogenic bone gel is very useful carrier for growth factors, which provides not only mechanical stability but also retains its osteo-inductivity, and anti-inflammatory activity. In order to apply allogenic bone gel to wider clinical practice we investigated the use of allogenic bone gel as a carrier for autologous bone-forming cells.

Allogenic bone gel was manufactured in line with Example 1. Bone marrow aspirates were obtained from the iliac crest of sheep. Multiple aspirations were carried out with variations of depths and punch points, using bone marrow aspiration needles (Angiotech, DBMNI 1501, Gainesville, Fla.) connected with 20-ml heparinised syringes. To prevent peripheral blood contaminating, no more then 2 ml bone marrow was harvested from each punch point. Approximate 10 ml total bone marrow was harvested per sheep. To remove red blood cells the buffy coat was centrifuged with ficoll-paque plus (Amersham Pharmacia Biotech, Uppsala, Sweden) at 1500 rpm for 10 mins, and nucleated cells were collected and the cell number was counted under light microscope.

The nucleated cells were then cultured with complete culture medium which composed of alpha minimum essential medium (α-MEM) supplemented with 10% heat-inactivated fetal bovine serum (FBS), 100 U/ml penicillin, 100 μg/ml streptomycin, and 2 mM L-glutamine (all from ThermoTrace, Melbourne, Australia). The re-suspended cells (1×10⁵ cells/ml) were plated in 75 cm² culture flasks and incubated at 37° C., 90% humidity and 5% CO₂.

After 48 h incubation, non-adherent cells were discarded, and adherent cells were washed with serum free medium twice and fresh complete medium was added into the flasks. The cells were then incubated for further 7-10 days before being collected. The medium was changed every two day. To expand cell numbers, dense cell plaques were trypsinised with 0.25% trypsin and 1 mM EDTA for 5 min at 37° C. The cells were then passaged into two 75 cm² flasks on a par and the same procedure was repeated once soon the cell population reaching 80% of flask capacity. The confluent monolayer of cells was collected after the second passage.

To stimulate osteogenic differentiation, the cells were further incubated for 2 weeks in standard medium supplemented with 2.0×10⁻¹ M ascorbic acid, 7×10⁻³ M beta-glycerophosphate, 1.0×10⁻⁸ M dexamethasone or BMP-2.

The MSCs prepared as above were then loaded onto the allogenic bone gel scaffold or milled bone in the density of 1.5×10⁴/scaffold (approximate 0.01 g). The scaffold or milled bone loaded with the cells was then spun down at 500 rpm for 5 min to enhance the attachment of the cells on to the scaffolds. The scaffolds with the cells were then cultured in standard medium supplemented with 1) 2.0×10⁻⁴ M ascorbic acid, 7×10⁻³ M beta-glycerophosphate and 1.0×10⁻⁸ M dexamethasone; or 2) BMP-2, in 48-well plates to stimulate osteogenic differentiation. The medium were changed every 2 days. Scaffolds with the cells were sampled at 1, 2, 3, or 4 weeks after co-culture commenced. The evaluations, including histological examination and RT-PCR were carried out to determine the growth and differentiation of MSCs' in allogenic bone gel and milled bone.

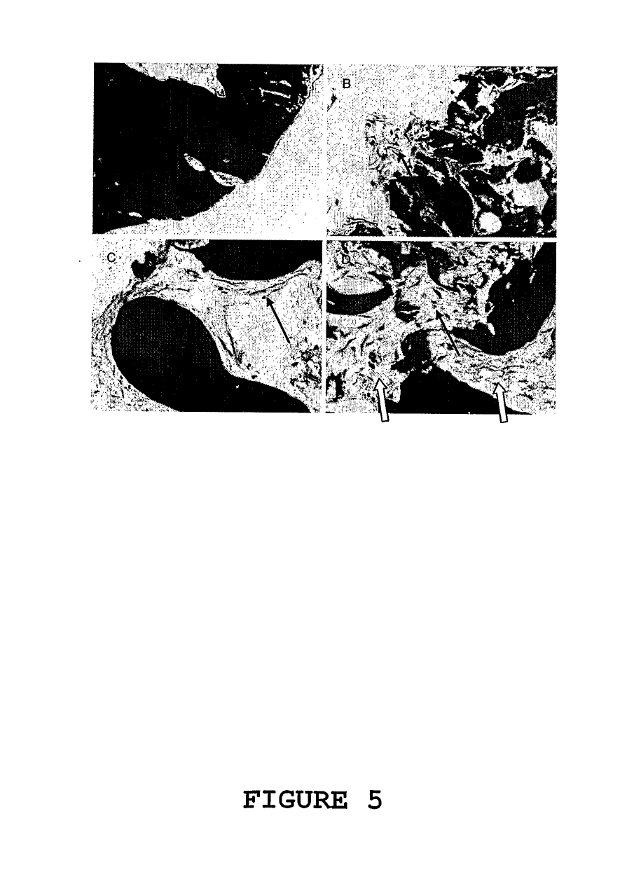

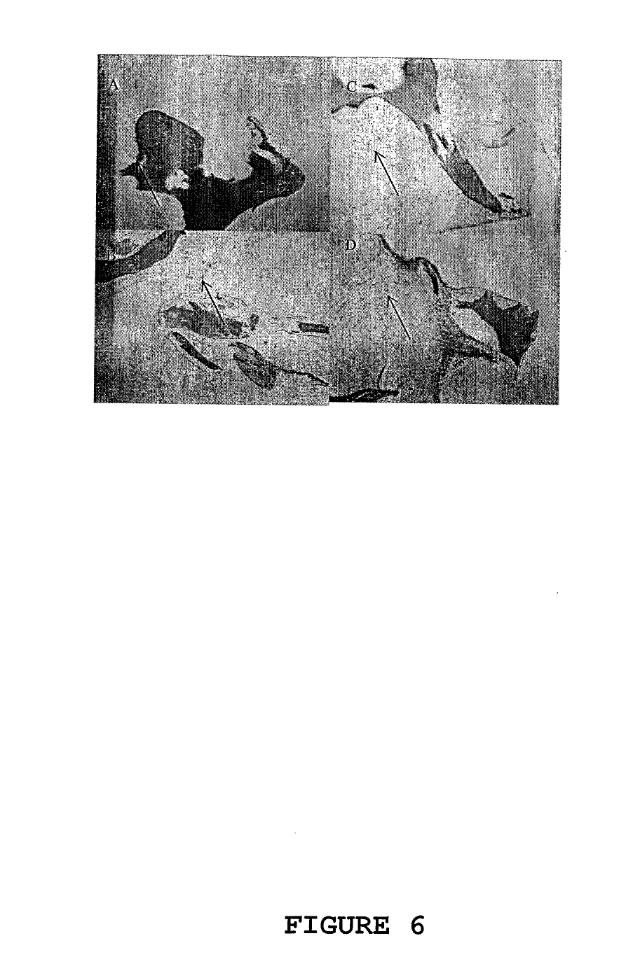

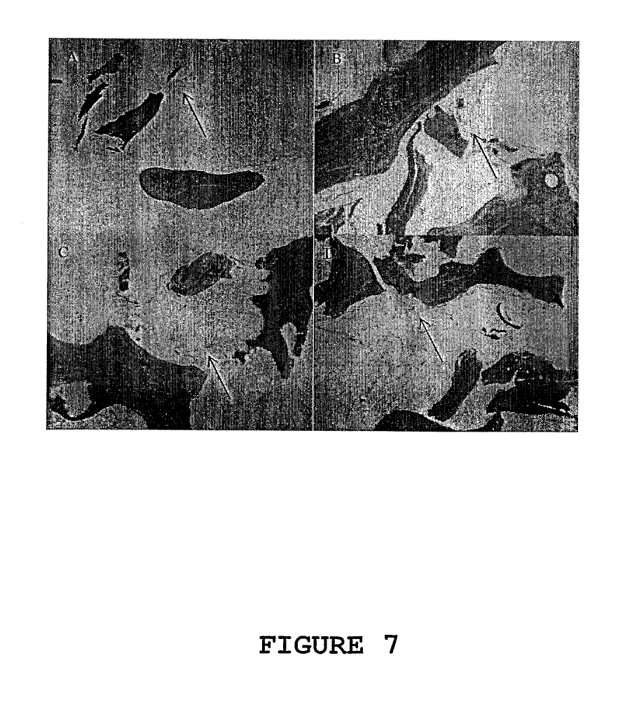

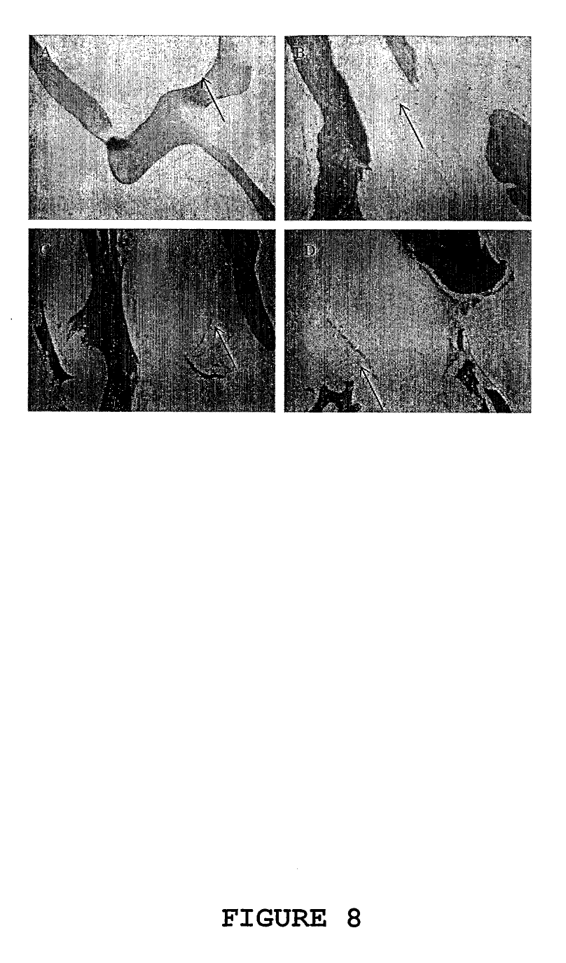

Histological analysis demonstrated that sheep MSCs (↑) grew in all allogenic bone gel scaffold and milled bone (FIGS. 5, 6, 7, and 8). In the first weeks, MSCs were observed attaching onto the surface of the scaffold, but not inside of scaffold (FIGS. 5A, 6A, 7A and 8A). The numbers of MSCs increased in weeks two and three and the cells started growing into the pores of the scaffold (FIGS. 5B, 5C, 6B, 6C, 7B, 7C, 8B and 8C). In week four, the cells filled the pores of the scaffold (FIGS. 5D, 6D, 7D and 8D).

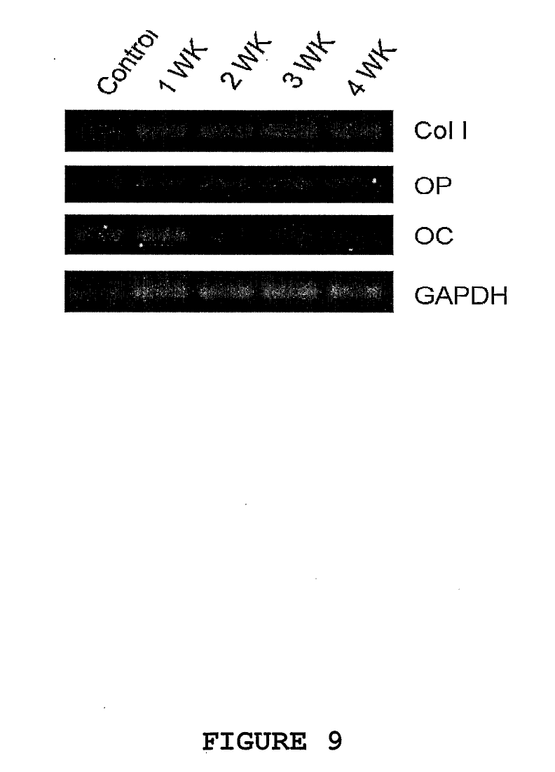

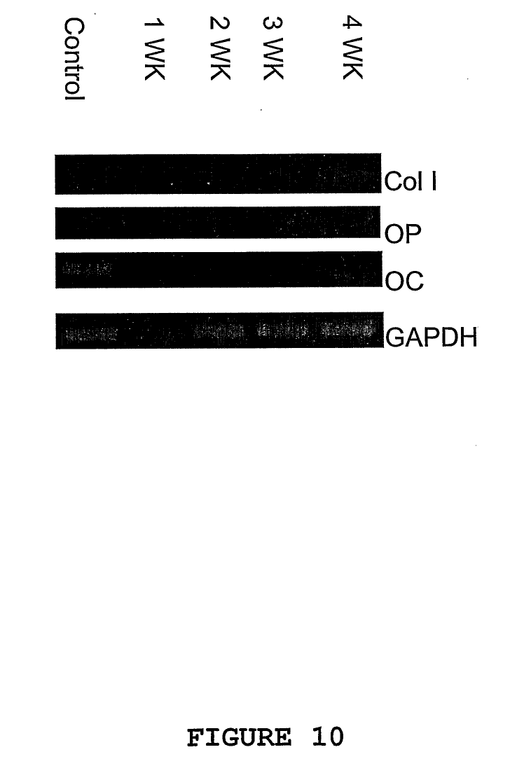

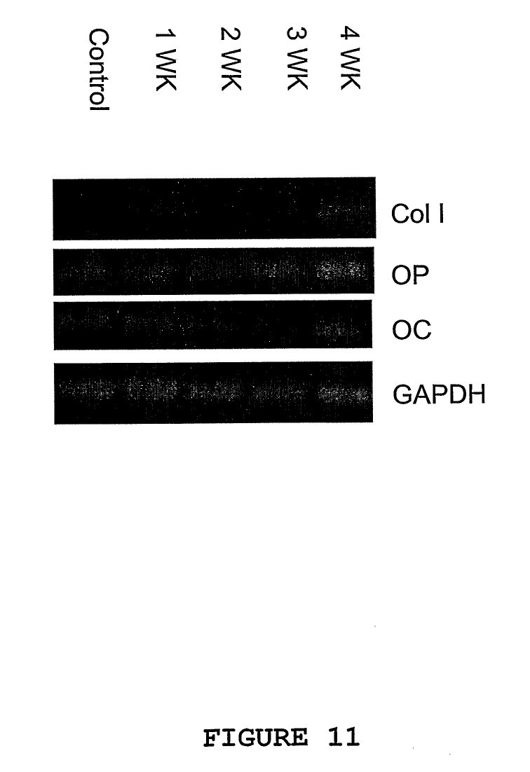

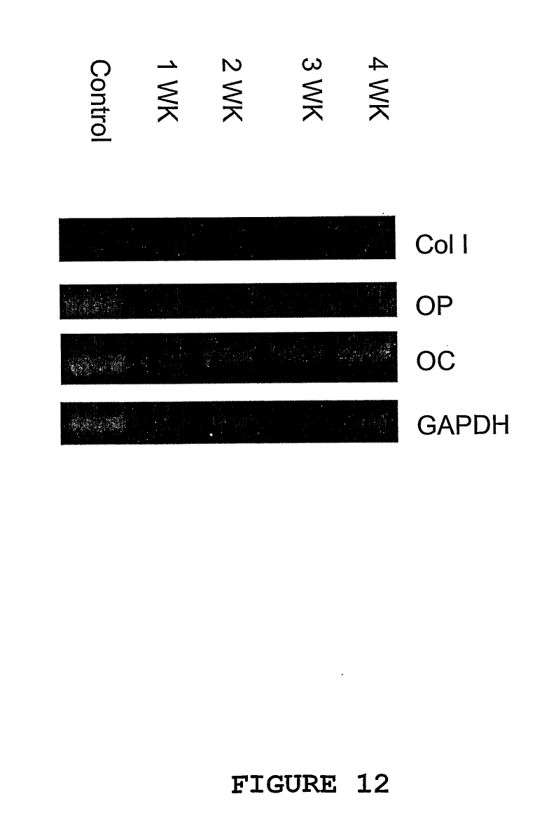

RT-PCR showed that osteoblast special genes including collagen type I (Col I), osteopontin (OP) and osteocalcin (OC) were expressed during co-culture of allogenic bone gel scaffold with MSCs/DXM or BMP-2 (FIGS. 9 and 11). OC expression, but not Col I or OP, was observed in some of samples cultures of milled bone with MSCs/DXM or BMP-2 (FIGS. 10 and 12).

The experiments demonstrated that when the bone forming cells, isolated from sheep bone marrow, cultured with allogenic bone gel scaffold in vitro, the cell number expended with time up to four weeks. The expression of osteoblast special genes including collagen type I, osteopontin and osteocalcin were detected during these times. Similar cell growth was observed when the bone forming cells were cultured with milled bone but only osteocalcin expression was detected in some of samples. The results indicated that allogenic bone gel as carrier for autologous bone forming cells provides a micro-environment enhancing the osteogenic differentiation of bone forming cells.

### Example 5

### Evaluation of Allogenic Bone Gel and Autologous Bone-Forming Cells In Vivo

32 female sheep about 55 kg were used for in this study. The sheep were housed in the Large Animal Facility of UWA for at least 10 days prior to surgery to allow for acclimatisation.

The sheep were premedicated with Acepromazine (0.05 mg/kg) combined with Buprenorphine (6-10 mcg/kg IM) approximately 20 minutes prior to induction. Anaesthesia was induced with a mixture of Diazepam and Ketamine used at a ratio of 1:1 (diazepam 5 mg/ml and ketamine 100 mg/ml; total of 1 ml per 10 kg approximately IV). They were then incubated and placed under Isofluorane (2% Isofluorane, O2 1 L/min) throughout the duration of the procedure. Vital signs were monitored throughout the entire operation. No complications were observed due to anesthesia during operation.

The sheep were randomised into four groups according to the material implanted: 1) autograft bone, 2) ABG, 3) autologous purified bone-forming cells (BFCs) alone, and 4) autologous BFCs intermixed with ABG. To investigate the induction of new bone formation, two postoperative time points, 6 and 10 weeks, were applied.

The sheep were positioned on their right lateral sides, and then routinely prepared, draped, and sterilized in a standard surgical fashion. Identification of vertebral levels was made by manual palpation of superficial landmarks using the sacrum as a reference. A paravertebral incision 15-20 cm long was made, and the spine was approached retroperitoneally from the plane between psoas minor in the midline.

### 1. Implantation of Autograft Bone and ABG

Following ligation of the segmental vessels, the intervertebral discs of L3-L4 and L4-L5 were removed. In group 1, autograft of cancellous bone harvested from the iliac crest earlier was implanted into the empty disc spaces. In group 2, ABG was implanted into the intervertebral discs of L3-L4 and L4-L5.

### 2. Bone Marrow Aspiration and Implantation

Immediately before the start of the surgical procedure, up to 25 ml of bone marrow was collected from the iliac crest area of the animals in Groups 3 and 4 using multiple small (2-ml) aspirates. To remove red blood cells the buffy coat was centrifuged with ficoll-paque plus (Amersham Pharmacia Biotech, Uppsala, Sweden) at 1500 rpm for 10 mins, and nucleated cells were collected and the cell number was counted under light microscope. 10⁵-10⁶ BFCs were then implanted into animals of group 3 or stirred into approximately 1 ml ABG before being implanted into the animals of group 4.

The vertebral bodies of L3-L4 and L4-L5 were linked up with bone staples (2 staples per segment) servicing as an internal fixation for stabilisation. The abdominal cavity was carefully investigated for any iatrogenic injury prior to closing. The wounds were closed with 1.0 absorbable suture to both the fascial and subcutaneous layers.

Postoperatively, the sheep were housed in floor pens without any restrictions to mobility or diet. No orthotic devices were applied. All sheep received prophylactic Amoxycillin (20 mg/kg IM) daily and Buprenorphine for analgesia (0.005˜0.01 mg/kg IM 4˜6 hourly) for 5 days.

### 3. Evaluation of Fusion Masses

Six or 10 weeks post operatively, the sheep were sacrificed and the spinal segments taken from L1 to the sacrum, stripped of soft tissue, and kept at −20° C. until further examination. The new bone formation was evaluated with micro CT scanning.

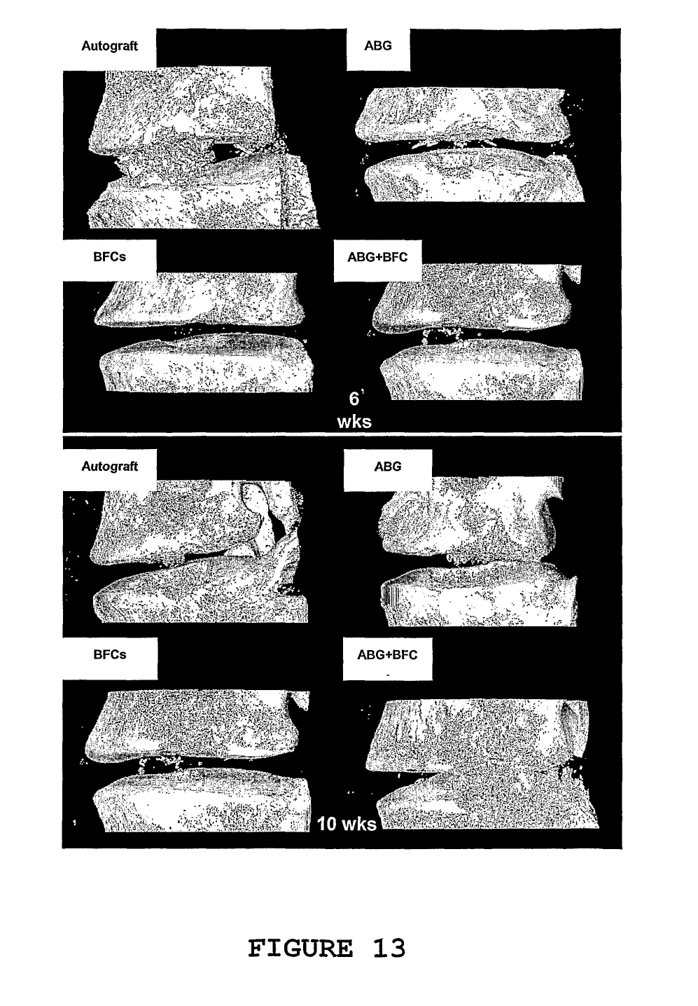

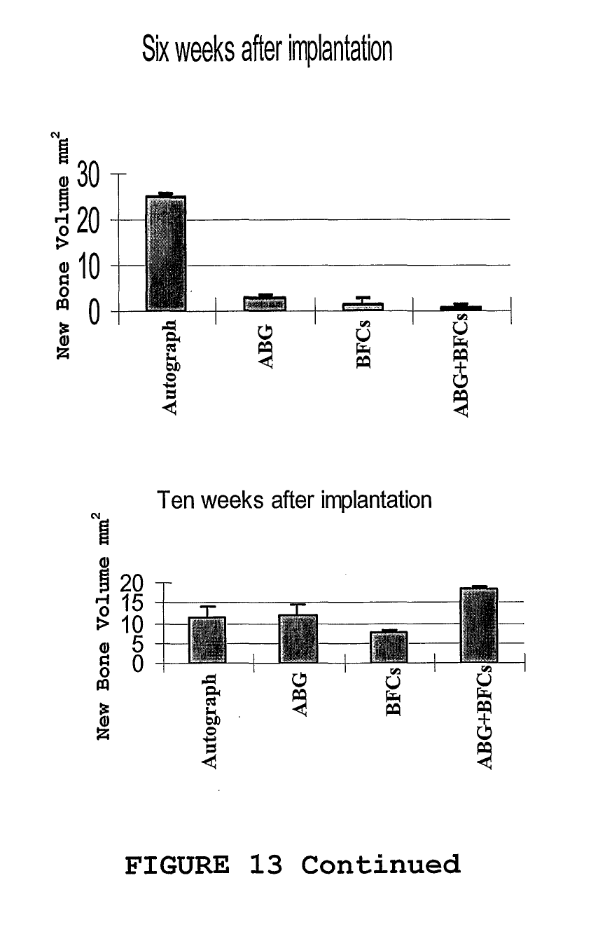

The new bone formation was discovered in all four groups six weeks after the operation (see FIG. 13). The most new bone formation (24.84 mm²) was found from the sheep implanted with autograft bone while the sheep implanted with BFCs+ABG showed least new bone formation (0.70 mm²). New bone formation was also found in the groups of the sheep implanted with ABG (2.56 mm²) and BFCs alone (1.13 mm²). 10 weeks after the operation, new bone formation was increased in three groups of the sheep implanted with ABG (11.91 mm²), BFCs (5.17 mm²) alone, and BFCs+ABG (18.17 mm²). New bone formation was decreased in the group of the sheep implanted with autograft bone (11.17 mm²).

It is clear from FIG. 13 that ABG can be used as an effective carrier for autologous BFCs in spinal fusion in a sheep model. These data also indicate that autologous bone forming cells and ABG induces more new bone formation than autograft bone, or either BFCs or ABG alone. Importantly, autologous bone forming cells and ABG not only induced new bone formation but also induced infiltration of the new bone into the existing bone strengthening the newly formed tissue.

It is thought that the superior bone formation produced by the combination of autologous bone forming cells and ABG can be attributed to the anti-inflammatory properties of ABG. As discussed elsewhere, inflammation associated with orthopaedic surgery is thought to promote bone loss. However, as demonstrated in the experiments discussed above, orthopaedic surgery employing autologous bone forming cells and ABG results in bone formation.

1. (canceled)

2. A bone forming carrier material comprising allogenic bone gel and a bone forming amount of bone forming cells, wherein the gel provides at least a 3 fold reduction in the number of polymorphonuclear leukocytes in a subject's tissue.

3. The bone forming carrier material of claim 2, wherein said bone-forming cells are selected from the group consisting of osteoblasts, mesenchymal stem cells and progenitor cells or combinations thereof.

4. The bone forming carrier material of claim 2, wherein said bone-forming cells are autologous cells.

5. The bone forming carrier material of claim 2, wherein the bone-forming cells are purified or substantially purified cells that express one or more genes selected from the group consisting of collagen type I, osteopontin and osteocalcin.

6. The bone forming carrier material of claim 2, wherein the bone-forming cells are purified or substantially purified osteoblasts.

7. The bone forming carrier material of claim 2, wherein said bone-forming cells comprise about 10⁵-10⁶ bone forming cells.

8. A medical device coated with the bone forming carrier material of claim 2.

9. A method of treating an orthopeadic condition comprising the step of administering the bone forming carrier material of claim 2.

10. A method of inducing bone formation in vivo comprising the steps of:
a. providing the bone forming carrier material of claim 2; and
b. applying said carrier material to a site requiring new bone formation.

11. A method of treating an orthopeadic condition comprising the step of administering the medical device of claim 7.
